(19) United States
(12) Patent Application Publication
(10) Pub. No.: US 2021/0292702 A1
(43) Pub. Date: Sep. 23, 2021

(54) LIVE CELL IMAGING CHAMBER AND MEASUREMENT THEREOF

(71) Applicant: UNIVERSITÉ LAVAL, Québec (CA)

(72) Inventors: Amine MILED, Québec (CA); Hamza LANDARI, Québec (CA)

(21) Appl. No.: 17/265,256

(22) PCT Filed: Aug. 1, 2019
(86) PCT No.: PCT/CA2019/051049

§ 371 Date: Feb. 2, 2021

Related U.S. Application Data
(60) Provisional application No. 62/716,453, filed on Aug. 9, 2018.
(60) Provisional application No. 62/716,028, filed on Aug. 8, 2018.

(51) Int. Cl. C12M 1/34; C12M 1/04; C12M 1/00; C12M 1/02; C12M 3/00; G01N 27/22; G01N 33/483; G02B 21/34; G02B 21/26

(52) U.S. Cl. CPC C12M 41/36; C12M 23/24; C12M 23/22; C12M 23/50; C12M 41/18; G02B 21/26; C12M 23/42; G01N 27/22; G01N 33/4833; G02B 21/34; C12M 23/34

(57) ABSTRACT

There is described a live cell chamber generally having a body having walls defining a sealed cavity, a sample area inside the cavity, a measurement device configured to perform one or more measurements on the sample area and a controller configured to generate a signal based on said one or more measurements. In one embodiment, the sealed cavity has a thicker inlet area and a thinner sample area, allowing to approach the focal plane of a microscope objective in the sample area. In one embodiment the sample area has a plurality of spaced-apart sample receiving regions having a hydrophilic material, and a sample repelling region surrounding each of the plurality of spaced-apart sample receiving regions and having a hydrophobic material. In another embodiment, a platform is provided to receive the sample in the cavity, and a provided mechanism allows movement between the top and bottom wall.

### BACKGROUND

Analysing live cells is required in various areas of life sciences and poses several challenges. This can require imaging the live cells and/or performing other measurements, while the live cells are supported in a liquid medium to form a liquid sample. It was known to use closed chambers to this end, chambers having a sealed internal cavity. One challenge in operating closed chambers is to preserve the culture environment during the analysis, which can require controlling the temperature and the atmosphere, while preventing contamination of the liquid sample by the surroundings. An additional challenge occurs when it is desired to perform a controlled administration of one or more liquids, such as performing a perfusion, to the sample. Such administration of liquids may need to be made in very small quantities, via microfluidics, for instance, and more than one type of liquid may need to be so administered.

Some existing techniques had been developed which were satisfactory to a certain degree. Indeed, it was known to provide closed chambers which were sealed from the environment except for gas and/or liquid inlets and outlets, sometimes referred to as ports, and which could be equipped with heating devices to preserve temperature. However, there always remains room for improvement.

### SUMMARY

One of the challenges was to provide the desired functionalities associated to managing the sample of live cells during imaging, while providing a chamber in a configuration which is adapted to most microscopes which are commonly used to perform cell imaging.

Indeed, uniform temperature in the chamber, and the minimization of fluctuations of temperature over time, are two concerns. On the other hand, the objective (lens assembly) of the microscope often needs to be approached close to sample. Indeed, the focal plane, which is at a given limited distance from the objective, is brought into coincidence with the position of the sample to obtain a clear image. To allow approaching the objective close to the sample, it can be desired to reduce the thickness of the chamber, in which the sample can lie on a slide which lays close to, or perhaps even forms, a bottom wall of the chamber, for instance, with a given volume of gas above it. However, reducing the thickness of the chamber in such circumstances led to the reduction of the volume of gas in the chamber above the sample, and it was found that this affected the ability to minimize temperature fluctuations temporally and spatially within the chamber. Indeed, when new gas is fed into the chamber by the chamber's corresponding inlet, the new gas may not have the same temperature as the controlled temperature of the culture environment formed by the volume of gas within the chamber. If the volume of gas within the chamber is proportionally large, the addition of a proportionally small volume of new gas via the inlet leads to mixing with relatively large volume of gas within the chamber and to a proportionally smaller temperature fluctuation. By contrast, if the volume of gas within the chamber is proportionally smaller, the same amount of new gas is mixed into a smaller volume of gas. This leads to a proportionally greater temperature fluctuation in the culture environment. Moreover, a smaller chamber volume can also restrict the mixing efficiency of the incoming gas, leading to hot or cool areas within the culture environment, another phenomenon which can be undesirable.

Two different solutions are proposed to address such issues. A first solution proposed is to use a chamber architecture which has a given, suitable thickness at its inlet area, to favor mixing in a relatively large volume of gas within the inlet area of the chamber, but which has a recessed section having a smaller thickness at a sample area in the chamber, forming an objective recess into which a microscope objective can be introduced to bring it closer to the sample.

The second solution is to provide the chamber with an adjustable-height platform acting as a sample holder. Indeed, the platform can have an adjustable height mechanism via which the sample can be brought relatively closer to, or farther away from, an observation wall of the chamber, similar to an internal “elevator” system onto which a microscope slide can rest, for instance. The microscope objective can be brought as close as possible to the observation wall of the chamber, and if in this position, the focal plane still does not reach the sample area, the mechanism can be activated to bring the platform closer to the microscope objective, and thereby bring the sample into coincidence with the focal plane, for instance.

In accordance with one aspect, there is provided a live cell imaging chamber having a chamber body having a sealed cavity between a bottom wall and a top wall, the sealed cavity having an inlet area and a sample area, the chamber body further comprising an inlet leading into the inlet area, and an outlet, the inlet area being thicker, between the bottom wall and the top wall, than the sample area to favor diffusion and mixing of gas received in the sealed cavity from the inlet, the top wall having an optically transparent window allowing imaging access to a sample positioned adjacent the bottom wall in the sample area, wherein the chamber body is thinner at the sample area than at the inlet area to allow approaching a microscope objective to the sample.

In accordance with another aspect, there is provided a live cell imaging chamber having a chamber body having a sealed cavity between a bottom wall and a top wall, and a sample receiving platform inside the sealed cavity, the chamber body further comprising an inlet leading into the cavity, and an outlet, the top wall having an optically transparent window allowing imaging access to a sample received by the platform, and a mechanism operable to move the platform between the top wall and the bottom wall.

Another one of the challenges is to provide a live cell measurement system which can receive more than one liquid sample at once. By doing so, measurements can be performed on the multiple liquid samples while the chamber remains closed, which can help avoid the drawbacks associated to successively opening and closing of the chamber to replace one liquid sample by another, as it can negatively affect the stability of the temperature and of the atmosphere of the culture environment.

In accordance with another aspect, there is provided a live cell measurement system having: a chamber body having walls defining a cavity; a sample area inside the cavity, the sample area having a plurality of spaced-apart sample receiving regions having a hydrophilic material, and a sample repelling region surrounding each of the plurality of spaced-apart sample receiving regions and having a hydrophobic material in a manner that when one of the sample receiving regions is subjected to a liquid sample, the liquid sample is maintained on the one of the plurality of spaced-apart sample receiving regions and repelled by the surrounding sample repelling region; a measurement system configured to perform one or more measurements on the liquid sample maintained on each of the plurality of sample receiving regions; and a controller configured to generate a signal based on said one or more measurements.

In some embodiments, different liquid samples of a same liquid sample source are receiving in each of the spaced-apart sample receiving regions. For instance, different liquid samples can be deposited in each of the sample receiving regions using a same pipette, which in this case acts as the liquid sample source. In alternate embodiments, the liquid sample source can be a tap or a bucket. In these examples, when the sample area is subjected to a liquid flow from the tap or when the sample area is dipped in the liquid content of the bucket, the liquid remaining on the sample area will be repelled by the sample repelling region towards the sample receiving regions, thereby providing a plurality of spaced-apart liquid samples ready for optical and/or electrical measurements.

However, it is noted that different liquid samples from different liquid sample sources may be received in the spaced-apart sample receiving regions. For instance, these different liquid samples can be deposited in the sample receiving regions using different liquid sample sources and/or pumping system(s). In these embodiments, using the live cell measurement system described herein can be convenient as it can prevent contamination between adjacent liquid samples, as they are maintained separate by the hydrophobic material of the sample repelling region.

Moreover, providing the configuration of hydrophilic material and hydrophobic material discussed above can cause the liquid samples maintained on the sample receiving regions to exhibit a spherical dome shape which maintain its shape over time and which can resist to movement of the sample area.

It will be understood that the expression “computer” as used herein is not to be interpreted in a limiting manner. It is rather used in a broad sense to generally refer to the combination of some form of one or more processing units and some form of memory system accessible by the processing unit(s).

It will be understood that the various functions of a controller can be performed by hardware or by a combination of both hardware and software. For example, hardware can include logic gates included as part of a silicon chip of the processor. Software can be in the form of data such as computer-readable instructions stored in the memory system. With respect to a computer, a controller, a processing unit, or a processor chip, the expression “configured to” relates to the presence of hardware or a combination of hardware and software which is operable to perform the associated functions.

Many further features and combinations thereof concerning the present improvements will appear to those skilled in the art following a reading of the instant disclosure.

### DESCRIPTION OF THE FIGURES

In the figures,

FIG. 1A is a side elevation view of a live cell imaging chamber in accordance with a first embodiment;

FIG. 1B is an oblique sagittal cross-sectional view of the live cell imaging chamber of FIG. 1A;

FIG. 2A is an oblique sagittal cross-sectional view of a live cell imaging chamber in accordance with a second embodiment;

FIG. 2B is an oblique view of the live cell imaging chamber of FIG. 2A, with a top wall made transparent;

FIGS. 2C and 2D are two oblique views of the live cell imaging chamber of FIG. 2A, with an objective distal therefrom, and proximal thereto, respectively;

FIGS. 3A and 3B show an alternate embodiment of a live cell imaging chamber wherein the sample is received in a cylindrical cell; and

FIG. 4 shows an alternate embodiment of a live cell imaging chamber wherein the bottom wall is formed by a circuit board to which a body portion of the chamber is clipped.

FIG. 5 is a sagittal cross-sectional view of a live cell imaging chamber in accordance with a third embodiment;

FIG. 6A is an oblique sagittal cross-sectional view of an example of a live cell measurement system configured for optical measurements, in accordance with an embodiment;

FIG. 6B is a top plan view of a sample area of the live cell measurement system of FIG. 6A, as taken along line 6B-6B of FIG. 6A;

FIG. 6C is a side elevation view of the sample area of FIG. 6B, taken along line 6C-6C of FIG. 6B;

FIG. 7 is a schematic view of another example of a live cell measurement system configured for electrical measurements, in accordance with an embodiment;

FIG. 8 is a schematic view of another example of a live cell measurement system, with a pumping assembly, in accordance with an embodiment;

FIG. 9 is a schematic view of an example of a computing device of a controller of the live cell measurement systems of FIGS. 6A and 7; and

FIG. 10 is a schematic view of a detailed example of a controller of the live cell measurement systems of FIGS. 6A and 7.

### DETAILED DESCRIPTION

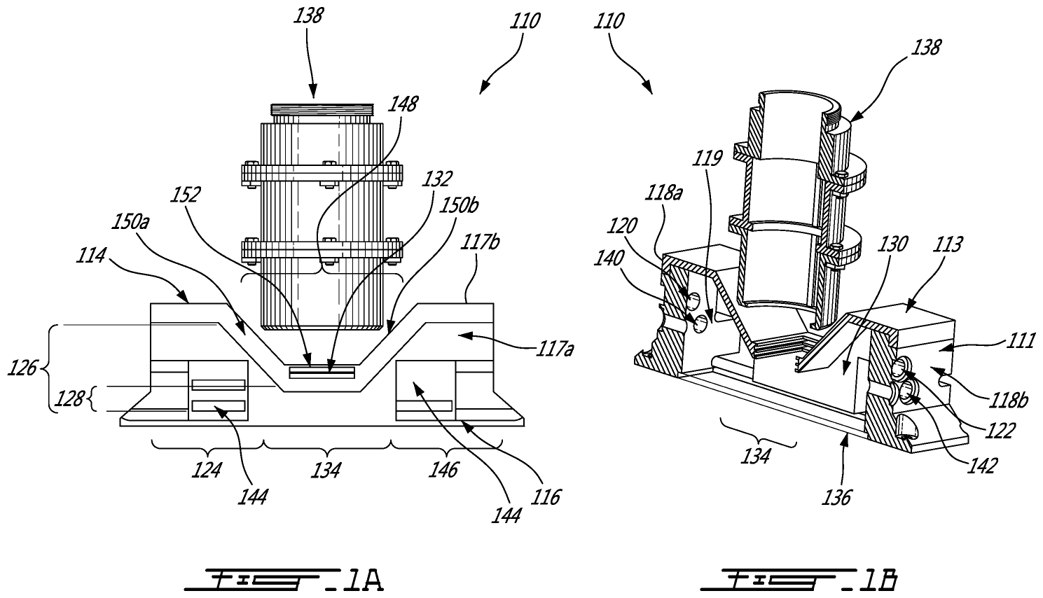

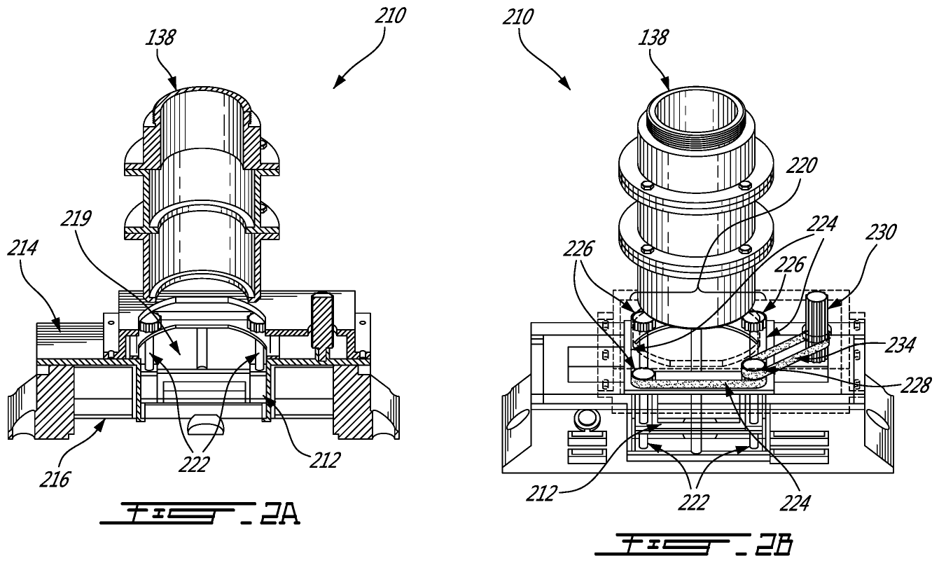

FIGS. 1A and 1B show a first example of a live cell imaging chamber 110. In this example, the live cell imaging chamber 110 has a chamber 110 comprised of a number of walls 114-118b and forming a sealed cavity 119 into which a controlled amount of gas can be introduced via an inlet 120 and an outlet 122. The inlet 120 leads into an inlet area 124 of the sealed cavity 119. The inlet area 124 of the sealed cavity is thicker 126, between the top wall 114 and the bottom wall 116, than a sample area 128, 134. This greater thickness 126 provides a greater volume of gas into which the gas introduced by the inlet 120 can more easily diffuse and mix, favoring a more uniform thermal and gaseous distribution for the culture environment 130 in the internal cavity 119.

An optically transparent window 132 is provided at the sample area 134 to provide optical access to the sample (i.e. transmission electromagnetic radiation within the optical region of the electromagnetic spectrum). The sample can be provided adjacent the bottom wall 116, such as on top of a microscope slide 136 laying on the bottom wall 116, for instance, to maximize the internal volume above the sample. The optically transparent window 132 can be a portion, or an entirety of the top wall 114, for instance. The top wall 114 has a uniform thickness, but an irregular shape, and the chamber 110 can be seen to conversely have a smaller thickness 128 at the sample area 134 than at the inlet area 124, 126. A microscope objective 138 can take advantage of this smaller thickness 128 to be engaged within a projection of the thickness of the inlet area 124 and approached to the sample, to align the focal plane with the sample, without preventing a satisfactory amount of gas diffusion in the inlet area 124.

In practice, such a live cell imaging chamber 110 will likely also be provided with one or more liquid (perfusion) inlet 140 and outlet 142, and a temperature control system which can include a temperature sensor for sensing the temperature in the sample area 134 and heating or cooling devices, such as Peltier devices 144 for instance, to control the temperature in the sample area 134 or more generally to control the temperature of the culture environment 130 within the cavity 119. Alternate embodiments can be provided with different options of culture environment control systems, and which can also include gas inlet(s) and outlet(s), for instance.

The chamber 110 can be said to have two opposite end walls 118a, 118b, and two opposite side walls 117a, 117b. Inlets 120, 140 and outlets 122, 142 can be provided through corresponding end walls 118a, 118b, for instance, whereas Peltier devices 144 can be provided in conjunction with metal plates to distribute the heat alongside walls 117a, 117b, for instance.

In this embodiment, it was chosen to provide the chamber 110 with a rectangular horizontal cross-section adapted to receive a typical 75 mm×25 mm microscope slide 136 on the bottom wall 116. As will be seen below, this is optional and there are other ways to receive a sample in the chamber 110.

The chamber 110 can be made of two sections, for instance, such as a body portion 111 and a cover portion 113, with the cover portion 113 being snugly engageable with the body portion 111 to form a tight seal, and removable therefrom to allow inserting the slide 136, and the sample, into the cavity 119, for instance. Alternately, one of the walls 114-118b of the chamber 110 can have an aperture to allow inserting the slide 136 thereacross in a tight, sealed fit manner, and the sample can then be introduced onto the portion of the slide 136 which extends in the cavity 119, via the open cover 113, for instance. Alternately, the chamber 110 can include a removable bottom portion and a body portion, and the sample can be introduced by removing the bottom portion rather than a cover portion, to name another example.

In some embodiments, especially when it is desired to maintain higher temperatures in the sealed cavity 119, it can be preferred that the walls 114-118b be provided with a certain amount of thermal insulation. In one embodiment, it can be preferred to use double walls of plastic, which can be 3D printed for instance with a spacing therebetween, and to blow an insulating material therebetween in a second step, for instance. In scenarios where apertures are provided across one or more of the walls 114-118b , conduits can be 3D printed between the double walls and also form a structure holding the double walls in a spaced-apart relationship to one another, for instance. Tubes for delivering fluid or drawing fluid from the cavity can then be connected to such apertures, for instance. Other forms of construction where double walls having with a thermal insulator sandwiched therebetween are possible.

In this embodiment, the chamber 110 is symmetrical and also has an outlet area 146 which is thicker 126 than the sample area 128, 134. The top wall 114 is formed with a V-shaped section 148 between the inlet area 124 and the outlet area 146, including two oppositely sloping wall sections 150a, 150b and a flat central portion 152 therebetween. The presence of sloping wall sections 150a, 150b, and especially the sloping wall section 150a between the inlet area 124 and the sample area 134, together with the positioning of the inlet 120, 140 and outlet 122, 142 at opposite ends 118a, 118b, can favor uniformity of the culture environment 130 by favoring smooth fluid flow between the inlet 120, 140 and the outlet 122, 142. In this embodiment, the optically transparent window 132 is a piece of glass inserted in the flat central portion 152. In an alternate embodiment, the sample area 134 can be lengthened between the inlet area 124 and the outlet area 146, and the window 132 can have an elongated rectangular shape, allowing to move the microscope objective 138 along the length of the sample area 134, for instance.

In this embodiment, Peltier devices 144 are integrated to the side walls 117a, 117b to achieve the temperature control. There can be two Peltier devices 144 on one side wall 117a, 117b, or Peltier devices 144 on both side walls 117a, 117b for instance. The Peltier devices 144 can be applied to a high thermal conductivity wall such as an aluminum wall for instance in a manner that the heat is transferred efficiently from the Peltier devices 144 to the aluminum wall, and the aluminum wall tends to quickly and evenly distribute the heat across its entire surface. In an embodiment, it was preferred to form a lining formed of four rectangular walls arranged in a parallelepiped shape, including two side metal walls connected (e.g. brazed) at each end to a corresponding one of two end metal walls, and to simply slide the lining into the cavity when the cover 113 is removed, and to then replace the cover 113. Such a lining can be adhered or otherwise fixed into place if desired. The end metal walls of such a lining can be provided with apertures which coincide with the fluid apertures 120, 140, 122, 142 provided across the walls 118a, 118b when the lining is in the operating position.

In some embodiments, the chamber 110 can be designed to receive a sample on a microscope slide 136, in which case it can be advantageous to size the chamber 110 for use with standard microscope slides. Microscope slides are commonly provided in centimeter-scale dimensions. For example, a commonly available size of microscope slides is 75 mm×25 mm (about 3″ by 1″). However, other sizes are available and the size of chamber 110 can be specifically adapted either to receive a specific size of slides, or to receive a plurality of sizes of slides, for instance.

It can be preferred to provide the bottom wall 116 with an optically transparent window as well, as some microscopes allow imaging the sample from underneath, or both from above and from underneath. In one embodiment, the bottom wall 116 can be formed by snappingly engaging a microscope slide 136 bearing the sample, for instance. Alternately, the bottom wall 116 can be formed of a removable microscope slide 136, for instance. In some cases, the chamber 110 can be designed with an optically transparent window only in the bottom wall 116, and the top wall 114 or cover 113 can be non-transparent, for instance.

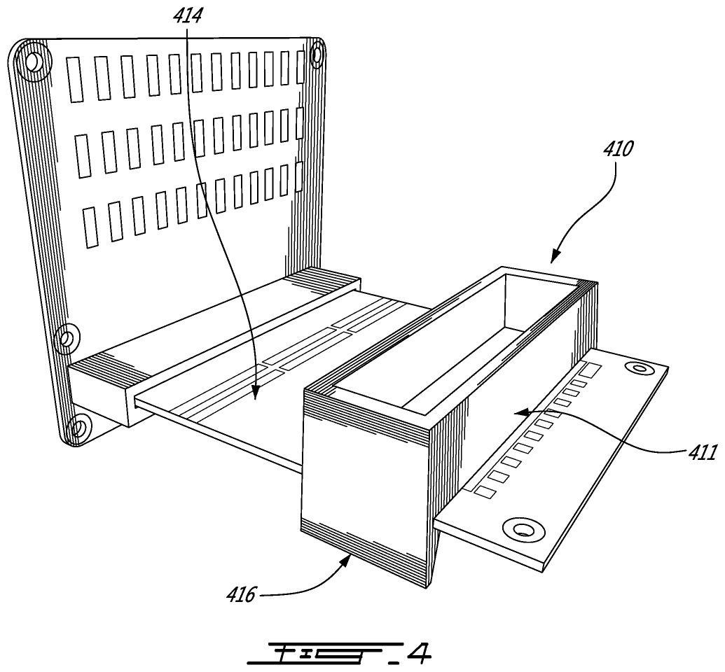

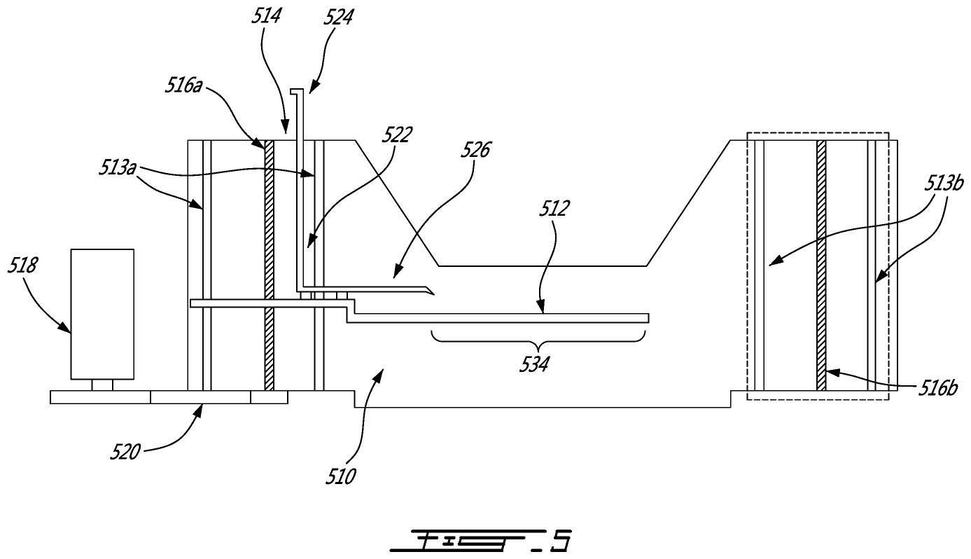

Turning now to FIGS. 3A and 3B, another embodiment is shown. In this embodiment, the chamber 310 also includes a body portion 311 and a cover portion 313. A plurality of cells 314 are integrated within the chamber 310 and into which samples are received. Each cell 314 has a cylindrical wall 328 extending perpendicularly upwards from the bottom wall 316. Each cell has an inlet 320 and an outlet 322 leading directly into the bottom portion of the cell 314 and allowing the introduction of liquids into, and extraction of liquid out from, the cell 314. In this embodiment, the body portion 311 and the cover portion 313 are both 3D printed and as shown more clearly in FIG. 3B, conduits 324 leading to the bottom portion of the cells 314 are provided as part of the structure of the body portion 311. The conduits 324 have a protruding portion 326 protruding outwardly from a corresponding one of the walls 317a, 317b, 318a, 318b and onto which a flexible tube can be sealingly mounted, for instance. An embodiment such as shown in FIGS. 1A and 1B can be modified to be provided with cells in a similar manner, for instance, instead of being designed to receive a sample on a microscope slide 136.

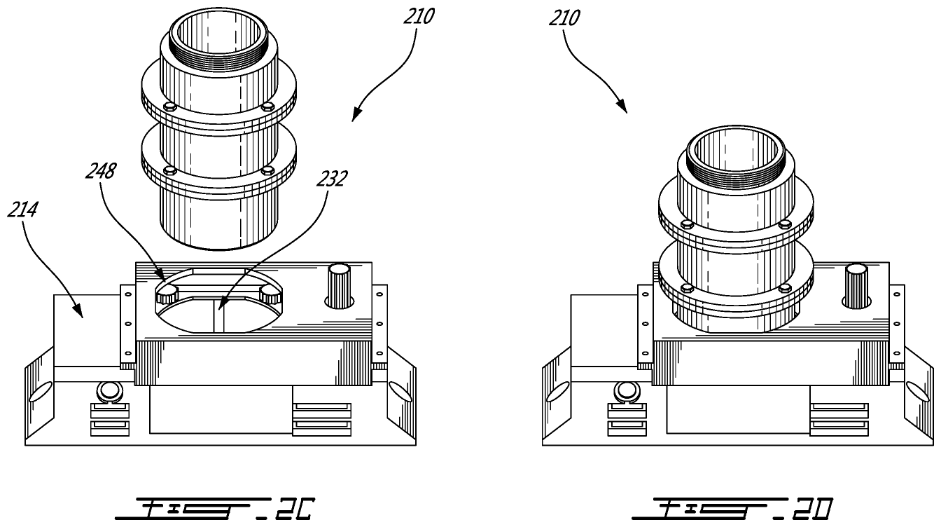

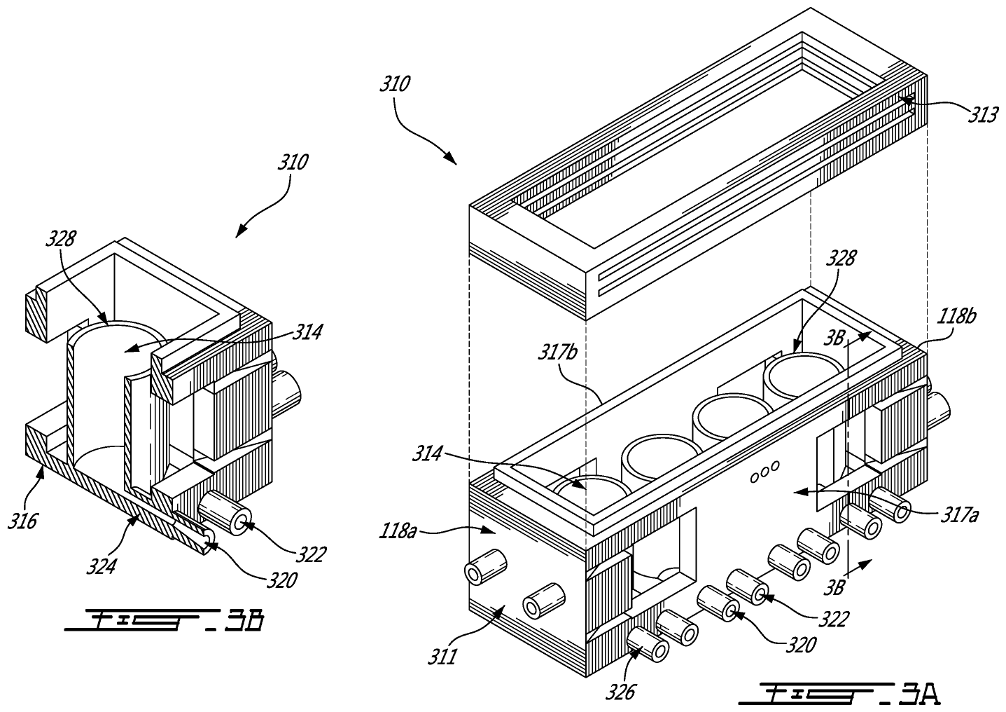

Turning now to FIGS. 2A to 2D, another embodiment of a live cell imaging chamber 210 is presented. In this alternate embodiment, the top wall 214 is generally planar and the thickness of the sealed cavity 219 is generally uniform between the top wall 214 and bottom wall 216. However, a platform 212 is provided to receive the sample (e.g. by receiving a microscope slide bearing the sample), and a mechanism 220 is provided to allow moving the platform 212 vertically between the top wall 214 and the bottom wall 216.

In this specific embodiment, the mechanism 220 includes a plurality of vertically-oriented endless screws 222 rotatingly mounted between the top wall 214 and the bottom wall 216. The platform 212 is threadingly engaged with the endless screws 222 in a manner that when the endless screws 222 rotate in a given angular direction, the platform 212 moves in a corresponding vertical direction between the top wall 214 and the bottom wall 216 (e.g. if turning in a right handed angular direction can raise the platform 212, whereas turning in the left handed angular direction can lower the platform 212). For convenience, the endless screws 222 can be made to rotate collectively. In this embodiment, this is achieved by connecting one end of the endless screws 222 to one another via a belt 224 and pulley 226 arrangement (miniaturized to adapt to the centimeter scale of the chamber 210). More specifically, the tips of each endless screw 222 can be provided with a corresponding pulley 226, and the pulleys 226 can be connected to one another via a belt 224.

To achieve satisfactory operability, it can be preferred to use at least three endless screws 222. In this specific embodiment, four endless screws 222 were used, forming a generally square belt 224 path. In this embodiment, the pulleys 226 and belt 224 protrude above the top wall 214, and the square belt 224 path was made to be adapted to forming an objective recess 248 into which an objective 138 can be engaged within the closed-loop of the belt 224 and approached to the optically-transparent window 232 of the top wall 214, such as shown in FIGS. 2C and 2D.

It can be desired to make the mechanism convenient to activate. This could be achieved by directly manipulating one pulley 228, for instance, via a knob 230 which protrudes vertically from the top wall 214. However, to avoid interference with the objective 138, it was preferred to offset the pivoting axis of the knob 230 from the objective recess area 248, and to this end, an additional belt 234 and pulley 228 arrangement, best seen in FIG. 2B, was used to convey the rotation of the knob 230 to the endless screws 222 and associate belt 224.

Turning now to FIG. 4, an example of a chamber 410 is provided where the bottom wall of the chamber 410 is not integrated to a body portion 411 thereof, but rather formed by a circuit board 414, and the body portion 411 of the chamber 410 can be provided with clips 416 or other fastening means which can allow securing the body portion 411 of the chamber 410 to the circuit board 414. It will be understood that embodiments such as shown in FIGS. 1A and 1B, or in FIG. 2A to 2D can be adapted to a configuration where they are designed in a manner to be clipped onto a circuit board 414, rather than having an integrated bottom wall, for instance.

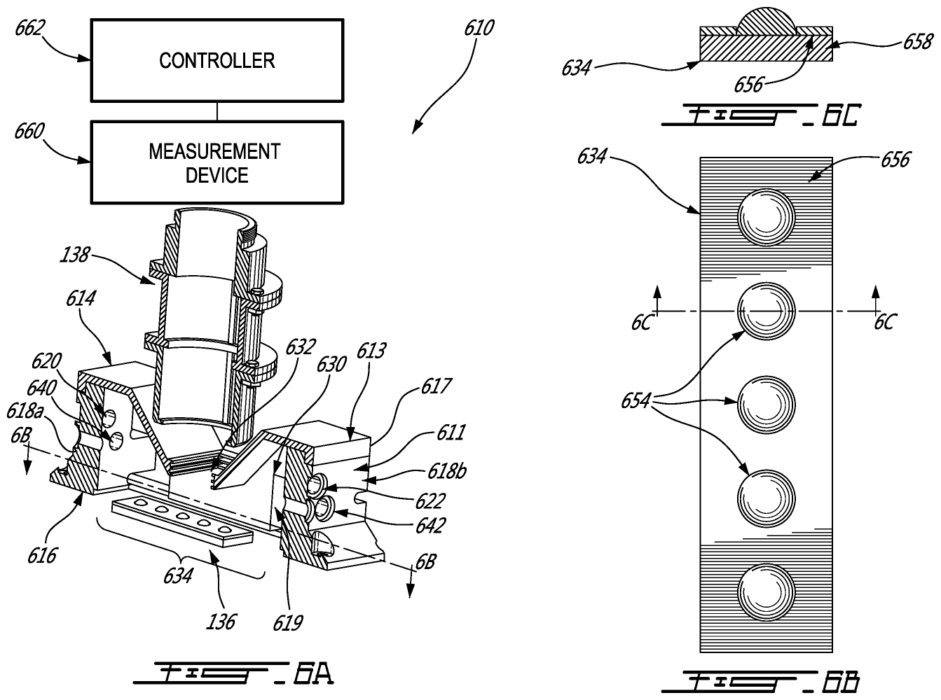

Many alternate configurations are possible. For instance, FIG. 5 shows one possible other embodiment where the platform 512 is mounted to slides 513a along which it can be raised or lowered by an endless-screw-type threaded rod 516a engaged with a female thread in the platform 512. In this embodiment, the actuator 518 is located adjacent to the chamber 510, and is connected to the threaded rod 516a via a gear transmission 520. One or more fluidic connection conduits 522 can be mounted to the platform 512 and have a rigid section 524 extending upwardly across an upper wall 514 of the chamber 510 via a sliding seal feature, in a manner to be raised and lowered along the sliding seal when the platform is raised or lowered. The end of the rigid section can be connected via flexible conduits, for instance. A horizontal segment of the conduit(s) 526 can bring the fluid(s) closer to the sample area 534, for instance. As schematized on the right hand side of FIG. 5, an optional slide 513b arrangement can be provided on both ends of the chamber 510 if desired, or only on one side if that is found sufficient. An additional threaded rod 516b, possible connected to a single actuator 518 via additional gears, a belt and pulley, or any suitable movement transfer mechanism, can also be provided on the opposite side if desired.

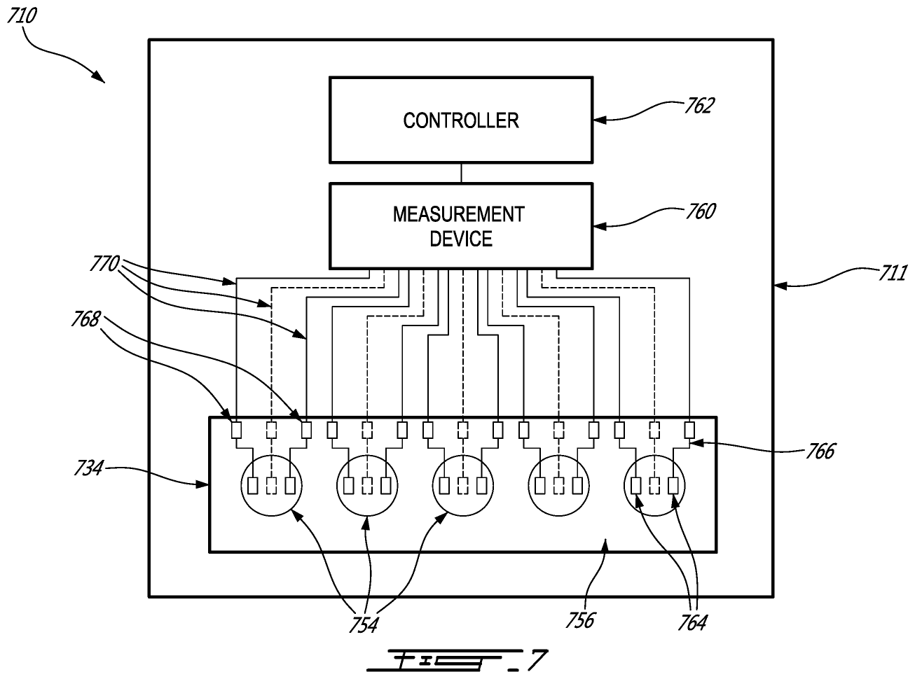

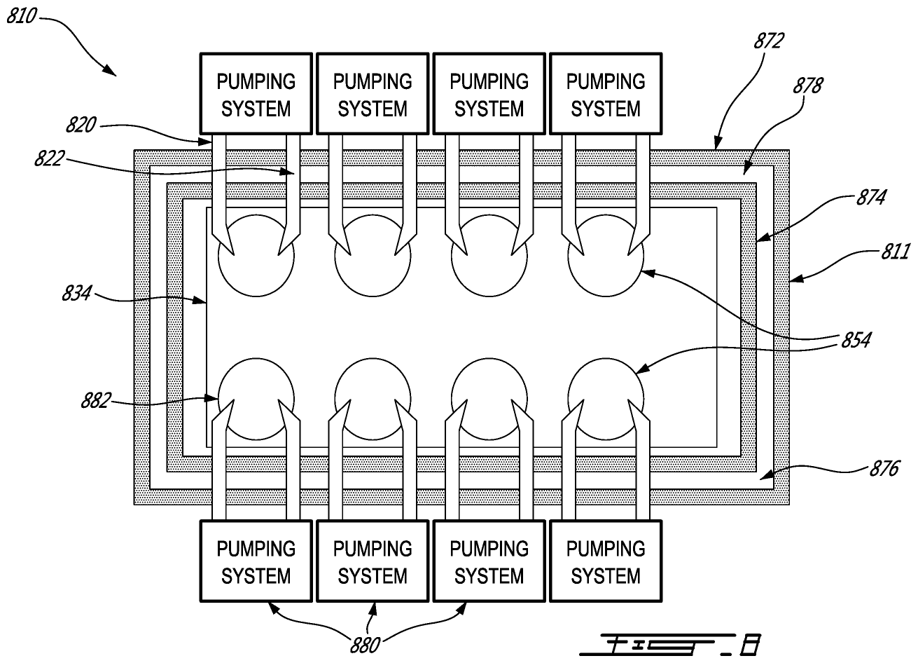

FIG. 6A shows an example of a live cell measurement system 610. As shown, the live cell measurement system has a chamber body 611 and cover 613 with a number of walls 614-618b defining a cavity 619. The system has a sample area 634 which is provided inside the cavity 619. More specifically, the sample area 634 has spaced-apart sample receiving regions 654, and a sample repelling region 656 surrounding each of spaced-apart sample receiving regions 654. It is emphasized that the sample receiving regions 654 have a hydrophilic material whereas the sample repelling region 656 has a hydrophobic material. For instance, in an embodiment, the sample area 634 includes a hydrophilic substrate 658 onto which hydrophobic material is disposed in a patterned manner to form the sample repelling region 656, and thus define the spaced-apart sample receiving regions 654. In this manner, when one of the sample receiving regions 654 is subjected to a liquid sample, the liquid sample is maintained on the corresponding sample receiving region 654 while being repelled by the surrounding sample repelling region 656. As can be understood, the sample area 634 can thus hold and maintain separate a plurality of spaced-apart liquid samples. As shown, the live cell measurement system 610 has a measurement device 660 which is configured to perform one or more measurements on the liquid samples and a controller 662 which is configured to generate a signal based on the performed measurement(s).

In this embodiment, the measurement device 660 is configured to perform optical measurements on the liquid samples maintained on the sample area 634. More specifically, in this specific example, a top one of the walls of the chamber 610 has an optically transparent window 632 allowing optical access to the sample area 634 to perform the optical measurement. The optically transparent window 632 can be a portion, or an entirety of the top wall 614, for instance. The type of optical measurement can differ from one embodiment to another. However, in this example, the optical measurement includes imaging using a microscope (only the objective of the microscope 138 is shown in FIG. 6A). In some other embodiments, other optical measurements such as Fourier transform infrared (FTIR) spectroscopy measurements can be performed on the liquid samples maintained on the sample area 634. Depending on the embodiment, these measurements can be performed sequentially, one liquid sample at a time or simultaneously, all the liquid samples at once.

It can be advantageous to size the chamber 610 for use with a standard microscope slide 136. Microscope slides are commonly provided in centimeter-scale dimensions. For example, a commonly available size of microscope slides is 75 mm×25 mm (about 3″ by 1″). However, other sizes are available (e.g., 75 mm×50 mm, 46 mm×27 mm, 48 mm×28 mm) and the size of chamber 610 can be specifically adapted either to receive a specific size of slides, or to receive a plurality of sizes of slides, for instance.

Referring now to FIGS. 6B and 6C, the sample area 634 is provided in the form of a microscope slide 136 which has been modified to act as the sample area 634. The microscope slide 136 has a glass substrate 658, which acts as the hydrophilic substrate, and an apertured layer of hydrophobic material 656 is applied onto the hydrophilic substrate 658 to form the sample repelling region 654 and thus define the sample receiving regions 654. In the illustrated example, the sample receiving regions 654 are shaped as circular sports. However, the shape of the sample receiving regions 654 can differ from one embodiment to another.

In this specific example, the glass of the microscope slide 136, which is preferably treated for cell culture and/or functionalized for different applications, is the hydrophilic material. The hydrophobic material can be a layer of such as poly-chloro-tri-fluoroethylene (Honeywell Hydroblock P2000HS) or a layer of Teflon. In some embodiments, it can be envisaged to deposit a layer of Teflon on a layer of poly-chloro-tri-fluoroethylene to provide the sample repelling region 656.

Different methods of producing the sample area 634 can be used. For instance, in one example embodiment, a stencil is deposited on an hydrophilic substrate 658 (e.g., a microscope slide 136), where the stencil is sized and shaped to cover one or more regions and to expose one or more other regions. In this embodiment, a layer of hydrophobic material, such as Teflon or other polymer, is deposited all over the stencil. The sample area 634 is thus obtained by removing the stencil, whereby the covered region(s) become sample receiving region(s) 654 and the exposed region(s) become sample repelling region(s) 656. In another example embodiment, apertures are cut in a substrate such as vinyl substrate using a cutting machine. A layer of hydrophobic material is applied all over the vinyl substrate, prior or after said cutting. Then, the treated vinyl substrate is deposited over the hydrophilic substrate 658 (e.g., a treated microscope slide 136), thereby leaving sample receiving region(s) 654 and sample repelling region(s) 656.

It is preferred that the materials forming the sample area 634 be able to sustain high temperatures. For instance, as liquid samples containing live cells are generally at 37° C., the sample area 634 supporting such liquid samples are generally sterilized at high temperature (>150° C.) in an autoclave. In other embodiments, the sample area 634 can be made disposable.

It can be preferred to provide the bottom wall 616 with an optically transparent window as well, as some microscopes allow imaging the sample area from underneath, or both from above and from underneath. In this embodiment, the bottom wall 616 is snappingly removable to allow the introduction and removal of a microscope slide 136 bearing the sample area 634. Alternately, the bottom wall 616 can be formed of a removeable microscope slide 136, for instance. In alternate embodiments, the sample area 634 is permanently fixed inside the cavity 619.

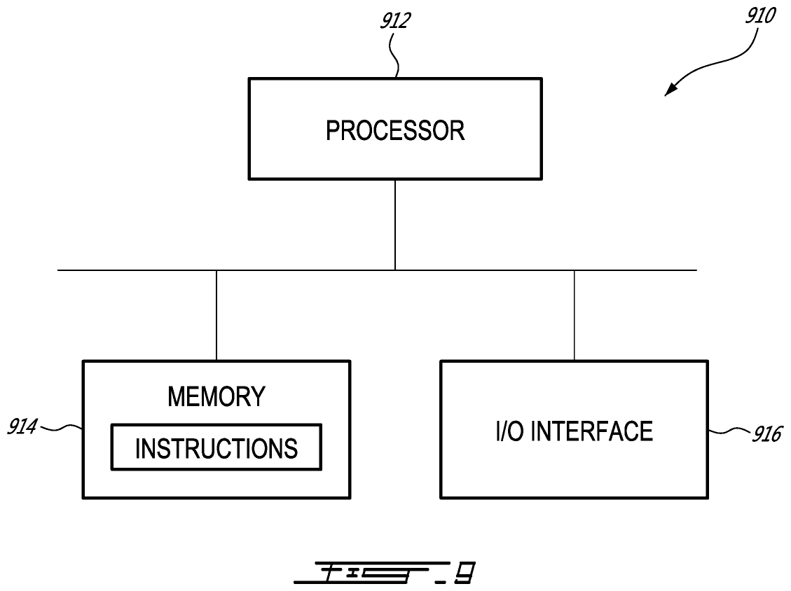

FIG. 7 shows another example of a live cell measurement system 710. In this specific example, the system 710 is configured for electrical measurements as the sample area 734 has two or more electrodes 764 being exposed to the liquid sample at each of sample receiving regions 754. As depicted, the measurement device 760 is electrically coupled to the electrodes 764 so as to perform electrical measurements on the liquid sample in each one of the sample receiving regions 754.

Typically, for capacitance and impedance measurements, only two electrodes 764 per sample receiving region 754 are required. However, for other types of electrical measurements, such as electrochemical measurements, three electrodes 764 per sample receiving region 754 are needed. The number of electrode 764 per sample receiving region 754 can thus depend on the embodiment. The electrodes 764 can be provided in the form of interdigital electrodes in some embodiments.

As shown in this example, the sample area 734 has conductive traces 766 connecting each electrode 764 to a respective conductive pad 768 proximate a periphery of the sample area 734. In this embodiment, the measurement device 760 is electrically coupled to the electrodes 764 via conductors 770, such as wires, electrically connected to the conductive pads 768. The wires 770 can be connected to the conductive pads 768 by any suitable connection technique. For instance, the conductive pads 768 can be connected to an interposer by wire bonding or alternately using conductive epoxy or conductive tapes.

Although not shown in FIG. 7, the conductive wires 770 can lead to an electrical connector which is removably connectable to the periphery of the sample area 734, thus connecting the wires 770 to the pads 768. In these embodiments, changing a sample area 734 for another can be made more efficient.

The electrodes 764 can be made of gold, silver or any other suitable conductive material. However, in embodiments where both optical and electrical measurements are to be made by the measurement device 760, it may be preferable to provide the electrodes 764 in Indium thick oxide (ITO), as such electrodes 764 can be optically transparent while being electrically conductive.

In this embodiment, the electrodes 764 are sized and shaped so that they have a thickness which is below 200 nm, preferably of about 150 nm, so that large surface effects are reduced. As can be understood, the thickness of the electrodes 764 can vary depending on the embodiment or fabrication process.

As can be understood, the number of sample receiving regions 754 of the sample area 734 can vary from one embodiment to another. For instance, in the illustrated embodiment, five sample receiving regions 754 are shown. However, in other embodiments, the sample area 734 can have more than ten sample receiving regions 754, more than twenty sample receiving regions 754 and even more than thirty sample receiving regions 754. The sample receiving regions 754 can be sized and shape so as to receive a predetermined volume of liquid sample. In some embodiments, the sample receiving regions 754 have a diameter above about 3 mm, above 5 mm or above 10 mm. For instance, in this embodiment, each sample receiving region 754 can receive up to 250 mm³ of liquid sample. However, this volume can be smaller or higher depending on the application.

Referring back to FIG. 6, a controlled amount of gas can be introduced in the cavity via an inlet 620 and an outlet 622 to monitor the culture environment 630 inside the cavity 619. Moreover, the chamber body 611 can have a liquid inlet 640 leading into the cavity, and a liquid outlet 642. As such, the liquid sample can be flowed onto the sample area 634 from the inlet 640. In this way, the liquid sample is repelled away from the sample repelling region 656 thereby being divided into a plurality of liquid samples towards corresponding ones of the plurality of spaced-apart sample receiving regions 654 where said liquid samples can be maintained steady for measurements.

FIG. 8 shows another example of a live cell measurement chamber 810. As depicted in this embodiment, the sample area 834 has eight spaced-apart sample receiving regions 854 which are surrounded by an apertured layer of hydrophobic material. The measurement device 760, the controller 762, the electrodes 764, conductive traces 766, pads 768 and wires 770, such as described with reference to FIG. 7, can be present but are not shown for simplicity.

In this specific embodiment, the sample area 834 is enclosed in a chamber body 811 having an outside wall 872, an inside wall 874, and a cavity 876 between the outside wall 872 and the inside wall 874. As shown, the cavity 876 is filled with insulating material 878 to provide insulation to the interior of the chamber body 811.

Moreover, the live cell measurement chamber 810 has a pumping assembly for pumping in liquid samples on the sample receiving regions 854 and for pumping out liquid samples present on the sample receiving regions 854. More specifically, in this example, each sample receiving region 854 has its respective pumping system 880 which is flowingly coupled to the corresponding sample receiving region 854 via inlet 820 and outlet 822 tubes. As shown, the inlet 820 and outlet 822 tubes have a pointy end 882 by which liquid sample can be delivered to the sample receiving region 854 and then removed from the sample receiving region 854. Although a plurality of separate and independent pumping systems 880 are shown in this embodiment, it is intended that a single pumping system, having multiple channels, could be used as well.

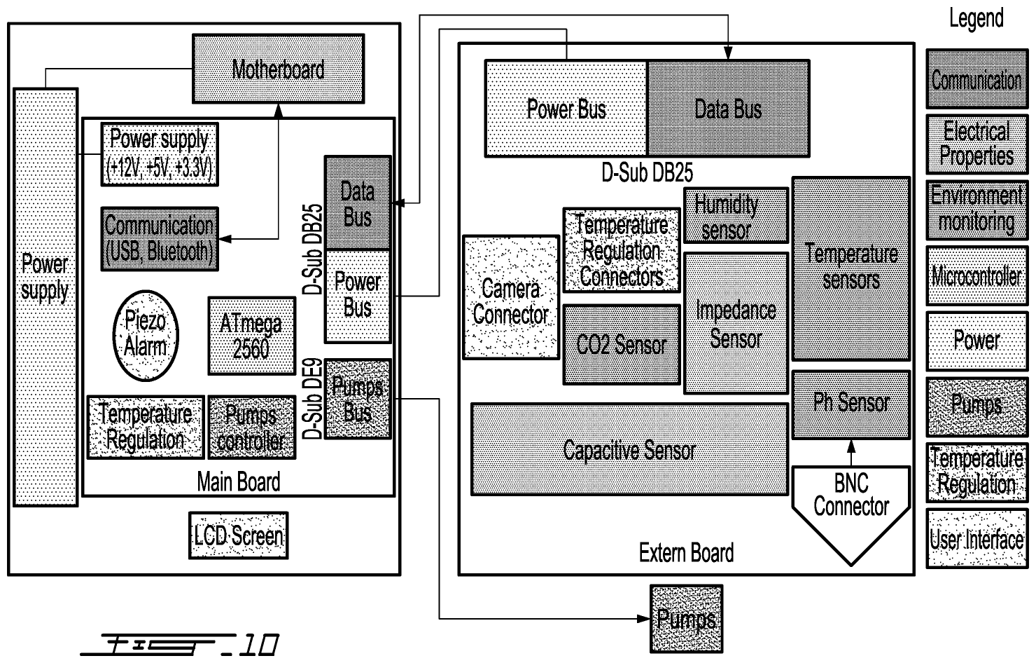

The controller can be provided as a combination of hardware and software components. The hardware components can be implemented in the form of a computing device, an example of which is described with reference to FIG. 9. Moreover, the software components of the controller can be implemented in the form of a software application in at least some embodiments.

Referring to FIG. 9, the computing device 910 can have a processor 912, a memory 914, and I/O interface 916. Instructions for generating the signal based on the performed measurements can be stored on the memory 914 and accessible by the processor 912.

The processor 912 can be, for example, a general-purpose microprocessor or microcontroller, a digital signal processing (DSP) processor, an integrated circuit, a field programmable gate array (FPGA), a reconfigurable processor, a programmable read-only memory (PROM), or any combination thereof.

The memory 914 can include a suitable combination of any type of computer-readable memory that is located either internally or externally such as, for example, random-access memory (RAM), read-only memory (ROM), compact disc read-only memory (CDROM), electro-optical memory, magneto-optical memory, erasable programmable read-only memory (EPROM), and electrically-erasable programmable read-only memory (EEPROM), Ferroelectric RAM (FRAM) or the like.

Each I/O interface 916 enables the computing 910 device to interconnect with one or more input devices such as the measurement device or with one or more output devices such as a display, a memory or the like.

Each I/O interface 916 enables the controller to communicate with other components, to exchange data with other components, to access and connect to network resources, to serve applications, and perform other computing applications by connecting to a network (or multiple networks) capable of carrying data including the Internet, Ethernet, plain old telephone service (POTS) line, public switch telephone network (PSTN), integrated services digital network (ISDN), digital subscriber line (DSL), coaxial cable, fiber optics, satellite, mobile, wireless (e.g. Wi-Fi, WiMAX), SS7 signaling network, fixed line, local area network, wide area network, and others, including any combination of these.

The computing device 910 described above is meant to be an example only. Other suitable embodiments of the controller can also be provided, as it will be apparent to the skilled reader. For instance, FIG. 10 shows a detailed example of the hardware of such a controller.

As can be understood, the examples described above and illustrated are intended to be exemplary only. For instance, in some embodiments, the cavity can enclose an optical subsystem including a plurality of movable reflective surfaces which can sequentially direct an optical beam towards each one of the sample receiving regions during the optical measurements. In these embodiments, the optical mechanism can be controlled. Such optical subsystem can include one or more collimating lenses to collimate the optical beam prior to reaching the liquid samples, and/or focusing lenses to focus the optical beam once it has propagated, and diffracted, through the liquid samples. The scope is indicated by the appended claims.

1. A live cell imaging chamber having a chamber body having a sealed cavity between a bottom wall and a top wall, the sealed cavity having an inlet area and a sample area, the chamber body further comprising an inlet leading into the inlet area, and an outlet, the inlet area being thicker, between the bottom wall and the top wall, than the sample area to favor diffusion of gas received in the sealed cavity from the inlet, the top wall having an optically transparent window allowing imaging access to a sample positioned in the sample area, wherein the chamber body is thinner at the sample area than at the inlet area to allow approaching a microscope objective to the sample.

2. The live cell imaging chamber of claim 1 wherein the top wall has a V-shaped section forming an objective-receiving recess above the sample, the V-shaped section having a flat central portion bearing the optically transparent window.

3. The live cell imaging chamber of claim 1 wherein the chamber body has a generally rectangular horizontal cross-section, has two end walls and two side walls.

4. The live cell imaging chamber of claim 3 wherein the inlet is in a first one of the two end walls, and the outlet is in a second, opposite one of the two end walls.

5. The live cell imaging chamber of claim 1 wherein the bottom wall further comprises an optically transparent window allowing reversed imaging access to the sample.

6. The live cell imaging chamber of claim 1 wherein the bottom wall is removable to allow introduction of a sample received on a centimeter-scale microscope slide.

7. The live cell imaging chamber of claim 6 wherein the microscope slide has 25 mm by 75 mm.

8. The live cell imaging chamber of claim 3 wherein the end walls and the side walls have double layers with a thermal insulator sandwiched between the double layers.

9. The live cell imaging chamber of claim 3 wherein aluminum plates are exposed in the cavity along each side wall, further comprising a Peltier device on a corresponding face of each aluminum plate, externally to the cavity.

10. A live cell imaging chamber having a chamber body having a sealed cavity between a bottom wall and a top wall, and a sample receiving platform inside the sealed cavity, the chamber body further comprising an inlet leading into the cavity, and an outlet, the top wall having an optically transparent window allowing imaging access to a sample received by the platform, and a mechanism operable to move the platform between the top wall and the bottom wall.

11. The live cell imaging chamber of claim 10 wherein the mechanism includes at least three endless screws mounted vertically between top wall and bottom wall, the platform being threadingly engaged with the endless screws in a manner that when the endless screws rotate in a given angular direction, the platform moves in a corresponding vertical direction between the top wall and the bottom wall.

12. The live cell imaging chamber of claim 11 wherein the three endless screws are rotatingly engaged with one another via belt and pulley arrangement for collective rotation, further comprising a manually rotatable knob extending externally to the sealed chamber and operable to drive the belt and pulley arrangement.

13. The live cell imaging chamber of claim 11 wherein the mechanism includes four endless screws engaged with one another via a belt and pulley arrangement, the belt surrounding an objective-receiving recess.

14. The live cell imaging chamber of claim 10 wherein the chamber body has a generally rectangular horizontal cross-section, has two end walls and two side walls.

15. The live cell imaging chamber of claim 14 wherein the inlet is in a first one of the two end walls, and the outlet is in a second, opposite one of the two end walls.

16. The live cell imaging chamber of claim 10 wherein the bottom wall further comprises an optically transparent window allowing reversed imaging access to the sample.

17. The live cell imaging chamber of claim 10 wherein the bottom wall is removable to allow introduction of a sample received on a centimeter-scale microscope slide.

18. The live cell imaging chamber of claim 17 wherein the microscope slide has 25 mm by 75 mm.

19. The live cell imaging chamber of claim 14 wherein the end walls and the side walls have double layers with a thermal insulator sandwiched between the double layers.

20. The live cell imaging chamber of claim 14 wherein aluminum plates are exposed in the cavity along each side wall, further comprising a Peltier device on a corresponding face of each aluminum plate, externally to the cavity.

21. A live cell imaging chamber having a chamber body having a sealed cavity between a bottom wall and a top wall, at least one sample cell including a cylindrical wall extending upwardly from the bottom wall, the top wall having an optically transparent window allowing imaging access to a sample positioned in the sample cell, and a pair of fluid conduits providing fluid flow communication between the sample cell and the outside of the cavity, across the chamber body.

22.-36. (canceled)
